(19) United States
(12) Patent Application Publication
(10) Pub. No.: US 2009/0270456 A1
(43) Pub. Date: Oct. 29, 2009

(54) Novel chemical compounds

(76) Inventors: Masaichi Hasegawa, Ibaraki (JP); Kazuya Kano, Ibaraki (JP); Masato Nakano, Ibaraki (JP); Mariko Yamabe, Ibaraki (JP)

Correspondence Address: Glaxosmithkline;Corporate Intellectual Property UW2220, P O Box 1539, King of Prussia, PA, 19406-0939, US

(21) Appl. No.: 10/585,676

(22) PCT Filed: Jan. 6, 2005
(86) PCT No.: PCT/US05/00303

§ 371 Date: Jul. 7, 2006

Related U.S. Application Data
(60) Provisional application No. 60/535,267, filed on Jan. 9, 2004.
(60) Provisional application No. 60/551,972, filed on Mar. 10, 2004.

(51) Int. Cl. A61K 31/427; C07D 277/38; C07D 417/10; C07D 417/04; C07D 417/14; A61K 31/426; A61K 31/4439; A61K 31/444; A61P 7/00; A61P 31/12; A61P 35/00

(52) U.S. Cl. 514/333; 548/194; 546/270.4; 546/256; 514/370; 514/342

(57) ABSTRACT

This invention relates to newly identified compounds for inhibiting hYAK3 and/or CK2 proteins and methods for treating diseases associated with the imbalance or inappropriate activity of hYAK3 and/or CK2 proteins.

### FIELD OF THE INVENTION

This invention relates to newly identified compounds for inhibiting hYAK3 and/or CK2 proteins and methods for treating diseases associated with the imbalance or inappropriate activity of hYAK3 and/or CK2 proteins.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphates. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79, 181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p16 (Serrano et al, Nature 1993: 366, 704), which binds to and inhibits CDK4. Deletions or mutations in p16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11, 4045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

Our U.S. Pat. No. 6,323,318 describes two novel human homologs of yeast YAK1 termed hYAK3-2, one protein longer than the other by 20 amino acids. hYAK3-2 proteins (otherwise reported as REDK-L and REDK-S in Blood, 1 May 2000, Vol 95, No. 9, pp 2838) are primarily localized in the nucleus. hYAK-2 proteins (hereinafter simply referred as hYAK3 or hYAK3 proteins) are present in hematopoietic tissues, such as bone marrow and fetal liver, but the RNA is expressed at significant levels only in erythroid or erthropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-G, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of hYAK3 proteins are expected to stimulate proliferation of cells in which it is expressed. More particularly, inhibitors of hYAK3 proteins are useful to treat or prevent diseases of the erythroid and hematopoietic systems, caused by the hYAK3 imbalance including, but not limited to, neutropenia; cytopenia; anemias, including anemias due to renal insufficiency or to chronic disease, such as autoimmunity, HIV or cancer, and drug-induced anemias; and myelosuppression.

Another serine/threonine kinase is casein kinase-2 (CK2). CK2 is probably the most pleiotropic member of the protein kinase family, with more than 200 substrates known. Unlike the great majority of protein kinases, which are tightly regulated enzymes, CK2 is endowed with high constitutive activity, a feature that underlies its oncogenic potential. On the other hand, the presence of many viral proteins among the target of CK2 indicates that CK2 is exploited by viruses to phosphorylate proteins essential to their life cycle, and may play a role in viral infections as well. (Pharmacology & Therapeutics 93, pp 159-168, 2002.) Thus inhibitors of CK2 are useful in the treatment or prevention of cancer and viral infections.

### SUMMARY OF THE INVENTION

In a first aspect, the instant invention relates a compound of the formula I, or a salt, solvate, or a physiologically functional derivative thereof

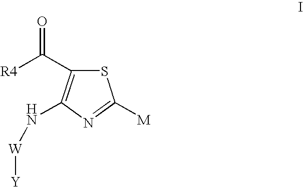

in which
M is a radical of the formula

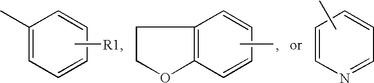

Y is a radical of the formula

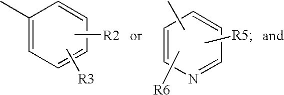

R1, R2 and R3 are independently hydrogen, —NH₂, halogen, —OC₁₋₆alkyl, —CF₃, —N(C═O)CH₃, —(C═O)OH, —CF₃, —(C═O)NH₂, —SO₂CH₃, —SO₂OH, or —C₁₋₆alkyl;
W is —(CH₂)ₙ—, in which n is 0 to 2; and
R4 is —NH₂, or —OH; and
R5 and R6 are independently hydrogen or halogen.

A preferred embodiment of Formula I compound is in which
M is a radical of the formula

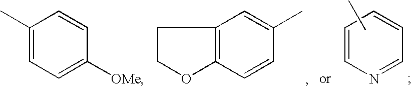

Y is a radical of the formula

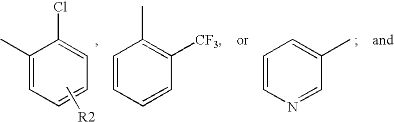

in which R2 is hydrogen, —NH₂, halogen, —OC₁₋₆ alkyl, —CF₃, —N(C═O)CH₃, —(C═O)OH, —CF₃, —(C═O)NH₂, —SO₂CH₃, —SO₂OH, or —C₁₋₆alkyl.

In a second aspect, the instant invention relates a method of inhibiting hYAK3 and/or CK2 in a mammal; comprising, administering to the mammal a therapeutically effective amount of a compound of the formula I, or a salt, solvate, or a physiologically functional derivative thereof.

In a third aspect of the present invention, there is provided a pharmaceutical composition including a therapeutically effective amount of a compound of formula I, or a salt, solvate, or a physiologically functional derivative thereof and one or more of pharmaceutically acceptable carriers, diluents and excipients.

In a fourth aspect of the present invention, there is provided the use of a compound of formula I, or a salt, solvate, or a physiologically functional derivative thereof in the preparation of a medicament for use in the treatment or prevention of a disorder of the erythroid and hematopoietic systems mediated by the imbalance or inappropriate activity of hYAK3 proteins, including but not limited to, neutropenia; cytopenia; anemias, including anemias due to renal insufficiency or to a chronic disease, such as autoimmunity, HIV or cancer, and drug-induced anemias; and myelosuppression.

In a fifth aspect, the present invention relates to a method of treating or preventing diseases of the erythroid and hematopoietic systems, caused by the hYAK3 imbalance or inappropriate activity including, but not limited to, neutropenia; cytopenia; anemias, including anemias due to renal insufficiency or to a chronic disease, such as autoimmunity, HIV or cancer, and drug-induced anemias; and myelosuppression; comprising administering to a mammal a therapeutically effective amount of a compound of formula I, or a salt, solvate, or a physiologically functional derivative thereof and one or more of pharmaceutically acceptable carriers, diluents and excipients.

In a six aspect, the present invention relates to a method of treating or preventing diseases selected from the group consisting of cancer; viral infections; neutropenia; cytopenia; anemias, including anemias due to renal insufficiency or to a chronic disease, such as autoimmunity, HIV or cancer, and drug-induced anemias; and myelosuppression; comprising administering to a mammal a therapeutically effective amount of a compound of formula I, or a salt, solvate, or a physiologically functional derivative thereof and one or more of pharmaceutically acceptable carriers, diluents and excipients.

In a seventh aspect of the present invention, there is provided the use of a compound of formula I, or a salt, solvate, or a physiologically functional derivative thereof in the preparation of a medicament for use in the treatment or prevention of cancer; viral infections; neutropenia; cytopenia; anemias, including anemias due to renal insufficiency or to a chronic disease, such as autoimmunity, HIV or cancer, and drug-induced anemias; and myelosuppression.

### DETAILED DESCRIPTION

The following terms may appear in the specification. If they appear, the following definitions will apply.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” refers to a straight or branched chain hydrocarbon. Furthermore, as used herein, the term “C₁₋₆ alkyl” refers to an alkyl group as defined above containing at least 1, and at most 6, carbon atoms. Examples of branched or straight chained “C₁₋₆ alkyl” groups useful in the present invention include methyl, ethyl, n-propyl, isopropyl, isobutyl, n-butyl, t-butyl, isopentyl, n-pentyl, n-hexyl, and the like.

As used herein, the term “halogen” refers to fluorine (F), chlorine (Cl), bromine (Br), or iodine (I).

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula I or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. Accordingly, the compounds of this invention include mixtures of enantiomers as well as purified enantiomers or enantiomerically enriched mixtures. Also included within the scope of the invention are the individual isomers of the compounds represented by formula I above as well as any wholly or partially equilibrated mixtures thereof. The present invention also covers the individual isomers of the compounds represented by the formulas above as mixtures with isomers thereof in which one or more chiral centers are inverted. Also, it is understood that all tautomers and mixtures of tautomers are included within the scope of the compounds of formula I.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula I. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate(embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula I, as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of compounds of the formula I and salts, solvates and physiological functional derivatives thereof, and one or more pharmaceutically acceptable carriers, diluents, or excipients. The compounds of the formula I and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula I, or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.6 mg to 1 g, preferably 1 mg to 700 mg, more preferably 5 mg to 100 mg of a compound of the formula I, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula I, and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula I, and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula I for the treatment of or prevention of diseases of the erythroid and hematopoietic systems, caused by hYAK3 imbalance or inappropriate activity including, but not limited to, neutropenia; cytopenia; anemias, including anemias due to renal insufficiency or to a chronic disease, such as autoimmunity, HIV or cancer, and drug-induced anemias; and myelosuppression; or for the treatment or prevention of diseases caused by CK2 imbalance or inappropriate activities, such as cancer or viral infections; will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula I per se. It is envisaged that similar dosages would be appropriate for treatment of the other conditions referred to above.

### Method of Preparation

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula I. Those skilled in the art will recognize if a stereocenter exists in compounds of formula I. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

More particularly, the compounds of the formula I can be made by the process of Scheme A or B. Any person skilled in the art can readily adapt the process of Scheme A or B, such the stoichemistry of the reagents, temperature, solvents, etc. to optimize the yield of the products desired.

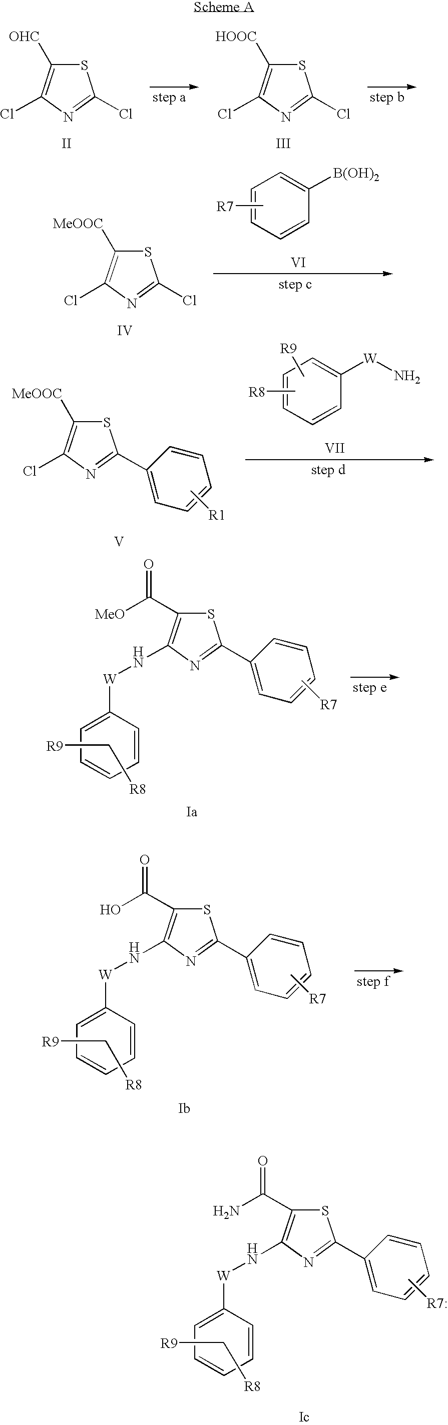

What follow are brief descriptions of the processes A and B.

In Scheme A, the compounds of formula II, III, IV and V can be synthesized as described in Example 1.

In step c, compound of formula IV, a compound of formula VI (about 1.1 equivalent), and tetrakis(triphenylphosphine)palladium(0) (about 0.05 equivalent) are suspended in a mixture of suitable solvent, such as DME, and 2M aqueous Na₂CO₃ under argon atmosphere. The mixture is heated at about 80° C. for about 5 h, and then poured into water. The resultant precipitate is filtered, washed with water, and dried under reduced pressure to give a compound of formula V.

In step d, a mixture of a compound of formula V, a compound of formula VII (about 1.5 equivalent), tris(dibenzylideneacetone)-dipalladium(0) or palladium (II) acetate (about 0.05 equivalent), sodium tert-butoxide (about 1.5 equivalent), tri-tert-butylphosphonium tetrafluoroborate or (R)-(+)-2,2′-bis(diphenylphosphino)-1,1′-binaphthyl (R—BINAP) (about 0.14 equivalent) are suspended in a suitable solvent, such as toluene, under argon atmosphere. The mixture is heated at about 100° C. for about 5 h, diluted with AcOEt, and washed with water. The organic phase is separated, concentrated in vacuo, and the residue is purified by chromatography on a silica gel column to afford a compound of Ia.

Step e is a typical hydrolysis step. To a solution of compound Ia in the mixture of THF and MeOH (about 5/2 ratio) was added 1N aqueous NaOH. The mixture is stirred at room temperature overnight, and then acidified with 1N HCl. The mixture is extracted with an organic solvent, such as CHCl₃, and then washed with water. The organic phase is separated and concentrated in vacuo to yield a compound of further within the scope of Formula I, i.e. formula Ib, which is typically purified by chromatography on a silica gel column.

In Step f, Sieber amide resin (700 mg 0.15 mmol) is treated with 20% piperidine in DMF (1 ml) in Alltech tube for 30 min. The mixture is drained and the resin is washed with DMF(3×). The mixture of a compound of formula Ib (0.076 mmol), HOBT (11 mg), HBTU (29 mg), DIEA (diisopropylethylamine, 0.027 ml) in DMF (1 ml) is added to the resin. The mixture is shaken at room temperature for about 15 h, at which point the liquid is drained. The resin is washed with DMF(3×), MeOH(3×), CH₂Cl₂(3×). It is dried under vacuum and treated with 10% TFA in CH₂Cl₂ (1 ml) for about 1 h. The solution is concentrated in vacuo to afford a compound further within the scope of Formula I, i.e. formula Ic, which is typically purified by chromatography on a silica gel column.

In Scheme A, R7, R8 and R9 are independently hydrogen, halogen, —OC₁₋₆alkyl, —CF₃, or —C₁₋₆alkyl; W is —(CH₂)ₙ—, in which n is 0 to 2.

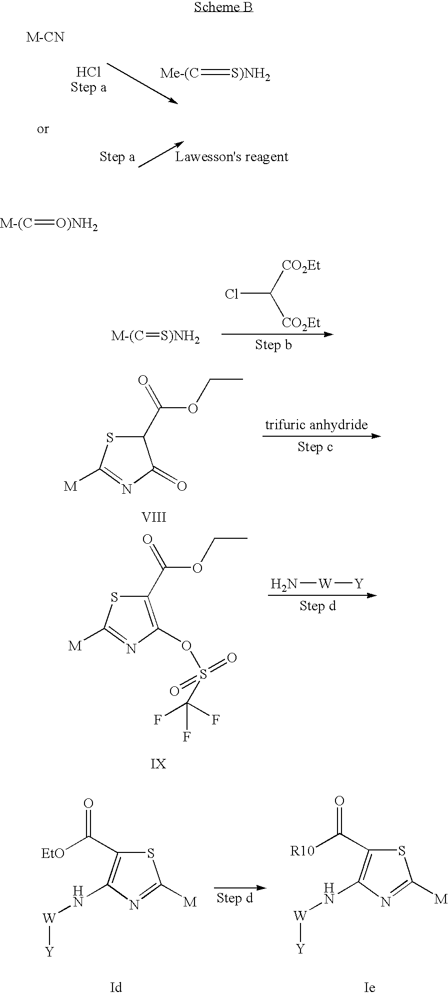

In Scheme B, a starting compound of formula VIII can be made by the standard reaction step b, as exemplified in Example 9. A compound of formula VIII is then reacted with trifuric anhydride in the presence of a base such as 2,6-di-t-butyl-4-methylpyridine to afford a compound of formula IX. A compound of formula IX is then coupled with an appropriate amine of the formula H₂N—W—Y in the presence of R—BINAP, Pd(OAc)₂, and CsCO₈ to afford a compound of formula Id. The ethyl ester group can further be hydrolyzed or replaced with NH₂ via a routine method in step d to afford an additional compound of formula Ie, within the scope of Formula I.

In Scheme A, M is a radical of the formula

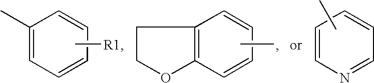

Y is a radical of the formula

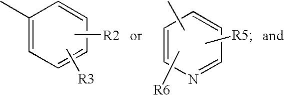

R1, R2 and R3 are independently hydrogen, —NH₂, halogen, —OC₁₋₅alkyl, —CF₃, —N(C═O)CH₃, —(C═O)OH, —CF₃, —(C═O)NH₂, —SO₂CH₃, —SO₂OH, or —C₁₋₆alkyl;
W is —(CH₂)ₙ—, in which n is 0 to 2; and
R10 is equal to —NH₂, or —OH; and
R5 and R6 are independently hydrogen or halogen.

### SPECIFIC EMBODIMENTS

### Examples

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); rt (room temperature);
min (minutes); h (hours);
mp (melting point); TLC (thin layer chromatography);
Tr (retention time); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); AcOEt (ethyl acetate);
DME (1,2-dimethoxyethane); DCM (dichloromethane);
DCE (dichloroethane); DMF (N,N-dimethylformamide);
DMPU (N,N′-dimethylpropyleneurea); (CDI (1,1-carbonyldiimidazole);
IBCF (isobutyl chloroformate); HOAc (acetic acid);
HOSu (N-hydroxysuccinimide); HOBT (1-hydroxybenzotriazole);
mCPBA (meta-chloroperbenzoic acid; EDC (ethylcarbodinimde hydrochloride); BOC (tert-butyloxycarbonyl);
FMOC (9-fluorenylmethoxycarbonyl); DCC (dicyclohexylcarbodiimide); CBZ (benzyloxycarbonyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine); BSA (bovine serum albumin)
ATP (adenosine triphosphate); HRP (horseradish peroxidase);
DMEM (Dulbecco's modified Eagle medium); HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluronium hexafluorophosphate).
HEPES (4-(2-hydroxyethyl)-1-piperazine ethane sulfonic acid);
DPPA (diphenylphosphoryl azide);
fHNO3 (fumed HNO3); and
EDTA (ethylenediaminetetraacetic acid).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (1R) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

HPLC were recorded on a Gilson HPLC or Shimazu HPLC system by the following conditions. Column: 50×4.6 mm (id) stainless steel packed with 5 □m Phenomenex Luna C-18; Flow rate: 2.0 mL/min; Mobile phase: A phase=50 mM ammonium acetate (pH 7.4), B phase=acetonitrile, 0-0.5 min (A: 100%, B: 0% O), 0.5-3.0 min (A: 100-0%, B: 0-100%), 3.0-3.5 min (A: 0%, B: 100%), 3.5-3.7 min (A: 0-100%, B: 100-0%), 3.7-4.5 min (A: 100%, B: 0%); Detection: UV 254 nm; Injection volume: 3 μL.

### Example 1

### 4-Anilino-5-carboxyl-2-(4-methoxyphenyl)thiazole (Iba)

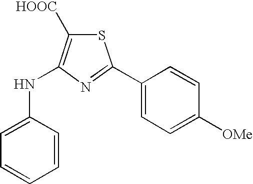

### 2,4-Dichloro-5-formylthiazole (II)

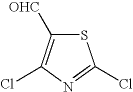

To a cooled solution of 2,4-thiazolidinedione (5 g, 42.7 mmol) in POCl₃ (24 ml) was added DMF (3.7 mL, 26.4 mmol). The mixture was stirred at room temperature for 1 hour, and then heated at 90° C. for 1 h and 115° C. for 4 h. The mixture was poured into a large amount of cold crashed ice. The mixture was extracted with CH₂Cl₂, washed with water, purified by chromatography on a silica gel column using CHCl₈ as an eluant to afford the title compound to afford compound II (3.6 g, 48%) as a brown solid. ¹H NMR (400 MHz, CDCl₃) ppm 9.90 (s, 1H).

### 5-Carboxyl-2,4-dichlorothiazole (III)

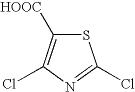

To a solution of compound II (500 mg, 2.74 mmol) in THF (18 ml) and water (12 ml) was added a mixture of sulfanylamide (346 mg, 3.56 mmol) and NaClO₂ (323 mg, 3.56 mmol) in water (2 ml). The mixture was stirred at room temperature for 2 hours, diluted with AcOEt, and washed with water. The organic phase was separated, concentrated in vacuo to afford the title compound of formula III (440 mg, 88%) as a yellow solid.

### 2,4-Dichloro-5-methoxycarbonylthiazole (IV)

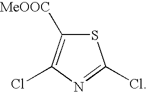

To a solution of compound III (1.04 g, 5.2 mmol), K₂CO₃ (1.078 g, 7.8 mmol) in DMF (20 ml) was added MeI (0.36 ml, 6.24 mmol). After stirring at room temperature for 5 hours, the mixture was diluted with AcOEt, and washed with water. The organic phase was separated, concentrated in vacuo, and then purified by chromatography on a silica gel column using CHCl₃ as an eluant to afford the title compound of formula IV (832 mg, 79%) as a pale yellow solid ¹H NMR (400 MHz, CDCl₃) ppm 3.91 (s, 3H)

### 4-Chloro-5-methoxycarbonyl-2-(4-methoxyphenyl) thiazole (Va)

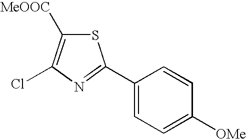

A mixture of compound IV (810 mg, 3.82 mmol), 4-methoxyphenyl-boronic acid (610 mg, 4.2 mmol), tetrakis(triphenylphosphine)palladium(0) (220 mg, 0.19 mmol) was suspended in the mixture of DME (20 ml) and 2M aqueous Na₂CO₃ (3.0 ml under argon atmosphere. The mixture was heated at 80° C. for 5 h, and then poured into water. (Normally a separate step of filtering through the Celite to remove tetrakis(triphenylphosphine)palladium (0) is required, but in this example, such step was not necessary.) The resultant precipitate was filtrated, washed with water, and dried under reduced pressure to give a compound of formula Va (580 mg, 52%) as a yellow solid. 1H NMR (400 MHz, CDCl₃) ppm 3.88 (s, 3H), 3.92 (s, 3H), 6.97 (m, 2H), 7.91 (m, 2H). LC/MS: m/z 284 (M+1)+.

### 4-Anilino-5-methoxycarbonyl-2-(4-methoxyphenyl) thiazole (Iaa)

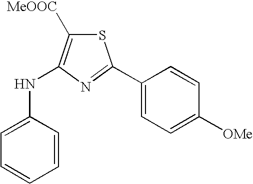

A mixture of compound Va (130 mg, 0.46 mmol), aniline (0.063 ml, 0.69 mmol), tris(dibenzylideneacetone)-dipalladium(0) (21 mg, 0.023 mmol), sodium tert-butoxide (60 mg, 0.69 mmol), tri-tert-butylphosphonium tetrafluoroborate (20 mg, 0.063 mmol) was suspended in toluene (1.0 ml) under argon atmosphere. The mixture was heated at 100° C. for 5 h, diluted with AcOEt, and washed with water. The organic phase was separated, concentrated in vacuo, and purified by chromatography on a silica gel column using CHCl₃ as an eluant to afford the title compound of formula Iaa (42 mg) as a yellow solid. ¹H NMR (400 MHz, CDCl₃) ppm 3.87 (s, 3H), 3.88 (s, 3H), 6.97 (m, 2H), 7.02 (m, 1H), 7.35 (m, 2H), 7.69 (m, 2H), 7.97 (m, 2H), 9.17 (s, 1H). LC/MS: m/z 341 (M+1)+.

### 4-Anilino-5-carboxyl-2-(4-methoxyphenyl) thiazole (Iba)

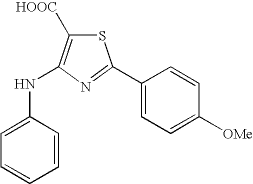

To a solution of compound Iaa (34 mg, 0.1 mmol) in the mixture of THF (0.5 ml) and MeOH (0.2 ml) was added 1N aqueous NaOH (0.1 ml). The mixture was stirred at room temperature overnight, and then acidified with 1N HCl. The mixture was extracted with CHCl₃, and then washed with water. The organic phase was separated, concentrated in vacuo, and purified by chromatography on a silica gel column using MeOH/CHCl₃ (0:1-1:10) as an eluant to afford the title compound of formula Iba (24 mg, 69%) as a yellow solid. 1H NMR (400 MHz, CDCl₃) ppm 3.88 (s, 3H), 7.00 (m, 2H), 7.11 (m, 2H), 7.35 (m, 2H), 7.68 (m, 2H), 7.99 (m, 2H), 9.28 (s, 1H). LC/MS: m/z 327 (M+1)+;

### Example 2

### 5-Aminocarbonyl-2-(3-methoxyphenyl)-4-(2-trifluoromethylanilino)thiazole (Ica)

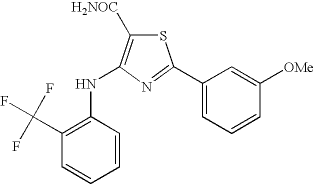

### 4-Chloro-5-methoxycarbonyl-2-(3-methoxyphenyl) thiazole (Vb)

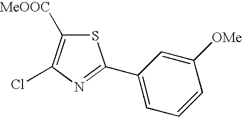

A mixture of compound IV (2.12 g, 10 mmol), 3-methoxyphenyl-boronic acid (1.67 g, 4.6 mmol), tetrakis(triphenyl phosphine)palladium(0) (578 mg, 0.23 mmol) was suspended in the mixture of DME (50 ml) and 2M aqueous Na₂CO₃ (8.0 ml) under argon atmosphere. The mixture was heated at 80° C. for 5 h, and then poured into water. The resultant precipitation was filtrated, washed with water, and dried under reduced pressure to give afford a compound of formula Vb (1.06 g, 43%) as a yellow solid 1H NMR (400 MHz, CDCl3) ppm 3.88 (s, 3H), 3.93 (s, 3H), 7.05 (m, 1H), 7.26 (m, 1H), 7.50 (m, 2H). LC/MS: m/z 284 (M+1)+.

### 5-Methoxycarbonyl-2-(3-methoxyphenyl)-4-(2-trifluoromethylanilino) thiazole (Iab)

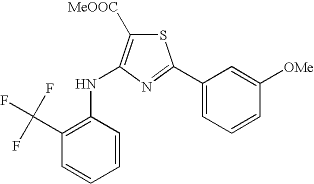

A mixture of compound Vb (200 mg, 0.7 mmol), 2-trifluoromethylaniline (0.13 ml, 1.05 mmol), tris(dibenzylideneacetone)dipalladium(0) (32 mg, 0.035 mmol), sodium tert-butoxide (51 mg, 1.05 mmol), tri-tert-butylphosphonium tetrafluoroborate (15 mg, 0.11 mmol) was suspended in toluene (1.0 ml) under argon atmosphere. The mixture was heated at 100° C. for 5 h, diluted with AcOEt, and washed with water. The organic phase was separated, concentrated in vacuo, and purified by chromatography on a silica gel column using CHCl₃ as an eluant to afford compound of formula lab (95 mg) as a yellow solid. LC/MS: m/z 409 (M+1)+.

### 5-Carboxyl-2-(4-methoxyphenyl)-4-(2-trifluoromethyl)aminothiazole (Ibb)

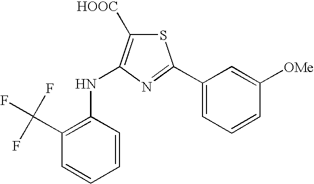

To a solution of compound Iab (95 mg, 0.28 mmol) in the mixture of THF (1.0 ml) and MeOH (0.5 ml) was added 2N aqueous NaOH (0.35 ml). The mixture was stirred at room temperature overnight, and then acidified with 1N HCl, and diluted with CHCl₃, washed with water. The organic phase was separated, concentrated in vacuo, and purified by chromatography on a silica gel column using MeOH/CHCl3 (0:1-1:10) as an eluant to afford the compound of formula Ibb (45 mg, 47%) as a yellow solid. ¹H NMR (400 MHz, CDCl₃) ppm 3.85 (s, 3H), 7.16 (m, 1H), 7.18 (m, 1H), 7.49 (m, 211), 7.60 (m, 1H), 7.72 (m, 2H), 8.44 (m, 1H), 9.28 (brs, 1H). LC/MS: m/z 395 (M+1)+;

### 5-Aminocarbonyl-2-(3-methoxyphenyl)-4-(2-trifluoromethylanilino)thiazole (Ica)

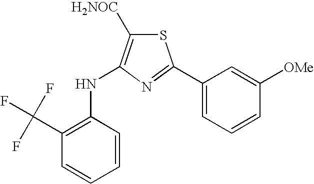

Sieber amide resin (700 mg 0.15 mmol) was treated with 20% piperidine in DMF (1 ml) in Alltech tube for 30 min. The mixture was drained and the resin was washed with DMF(3×). The mixture of 5-carboxyl-2-(4-methoxyphenyl)-4-(2-trifluoromethyl)anilinothiazole (Ibb) (30 mg, 0.076 mmol), HOBT (11 mg), HBTU (29 mg), DIEA (0.027 ml) in DMF (1 ml) was added to the resin. The mixture was shaken at room temperature for 15 h, at which point was drained. The resin was washed with DMF(3×), MeOH(3×), CH₂Cl₂(3×). It was dried under vacuum and treated with 10% TFA in CH₂Cl₂ (1 ml) for 1 h. The solution was concentrated in vacuo, and purified by chromatography on a silica gel column using CHCl₃ as an eluant to afford the title compound of formula Ica (7 mg, 23%) as a yellow solid. ¹H NMR (400 MHz, CDCl₃) ppm 3.88 (s, 3H), 5.38 (brs, 2H), 7.03 (m, 1H), 7.07 (m, 1H), 7.38 (m, 1H), 7.53 (m, 3H), 7.62 (m, 1H), 8.41 (m, 1H), 10.37 (brs, 1H). LC/MS: m/z 394 (M+1)+

The compounds in Examples 3-7 were obtained by process of Scheme A starting from 2,4-thiazolidinedione as in Example 1.

### Example 3

### 5-Carboxyl-4-(3-fluoroanilino)-2-(4-methoxyphenyl)thiazole (Ibc)

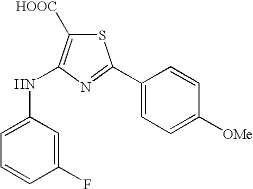

1H NMR (400 MHz, DMSO-d6) ppm 3.85 (s, 3H), 6.80 (m, 1H), 7.13 (m, 2H), 7.38 (m, 2H), 7.72 (m, 1H), 7.99 (m, 1H), 9.43 (brs, 1H). LC/MS: m/z 345 (M+1)+

### Example 4

### 5-Carboxyl-2-(4-methoxyphenyl)-4-(2-trifluoromethylanilino)thiazole (Ibd)

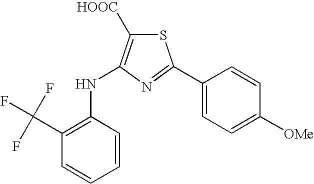

1H NMR (400 MHz, DMSO-d6) ppm 3.85 (s, 3H), 7.10 (m, 2H), 7.21 (m, 1H), 7.71 (m, 2H), 7.98 (m, 2H), 8.45 (m, 1H), 9.79 (brs, 1H). LC/MS: m/z 395 (M+1)+

### Example 5

### 4-Anilino-5-carboxyl-2-(3-methoxyphenyl)thiazole (Ibe)

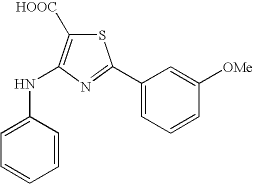

1H NMR (400 MHz, DMSO-d6) ppm 3.86 (8, 3H), 7.01 (m, 1H), 7.17 (m, 1H), 7.36 (m, 2H), 7.49 (m, 2H), 7.61 (m, 1H), 7.69 (m, 2H), 9.25 (brs, 1H). LC/MS: m/z 327 (M+1)+

### Example 6

### 5-Carboxyl-4-(2-fluoroanilino)-2-(3-methoxyphenyl)thiazole (Ibf)

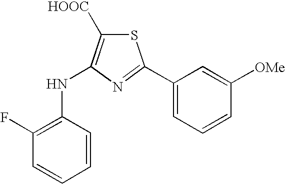

¹H NMR (400 MHz, DMSO-d6) ppm 3.86 (s, 3H), 7.00 (m, 1H), 7.16 (m, 1H), 7.27 (m, 2H), 7.48 (m, 1H), 7.51 (m, 1H), 7.62 (m, 1H), 8.46 (m, 1H), 9.75 (brs, 1H). LC/MS: m/z 345 (M+1)+

### Example 7

### 4-benzylamino-5-methoxycarboxyl 2-(4-methoxyphenyl)thiazole (Ibg)

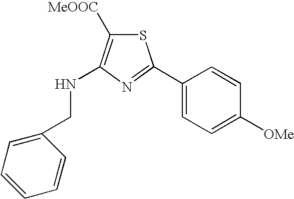

LC/MS: m/z 355 (M+1)⁺, retention time 4.95 min.

### Example 8

### 2-(2,3-Dihydro-benzofuran-5-yl)-4-oxo-4,5-dihydro-thiazole-5-carboxylic acid ethyl ester (VIIa)

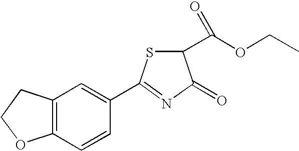

A mixture of 2,3-dihydrobenzofuran-5-thioamide (680 mg, 3.5 mmol) and diethyl 2-chloromalonate (630 mg, 3.5 mmol) in dry ethanol (15 mL) was heated at 80 degree for 5 hours. After cooling, the formed yellowish solid was collected by filtration and the solid was washed with ethanol to give the title compound (550 mg): LC-MS m/e 292 (M+1).

### 2-(2,3-dihydrobenzofuran-5-yl)-4-(trifluoromethanesulfonyloxy)thiazole-5-carboxylic acid ethyl ester (IXa)

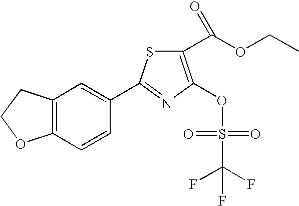

Under an argon atmosphere, to a mixture of 2-(2,3-dihydrobenzofuran-5-yl)-4-oxo-4,5-dihydrothiazole-5-carboxylic acid ethyl ester (500 mg, 1.72 mmol) and 2,6-di-t-butyl-4-methylpyridine (388 mg, 1.9 mmol) in CH₂Cl₂ (10 mL) was added dropwise trifuric anhydride (510 mg, 1.81 mmol) at 0 degree. Stirring was continued for 2 h at room temperature. The mixture was washed with H₂O and the product was extracted with CH₂Cl₂, dried over MgSO₄, and evaporated. Ether was added to the residue and the formed solid (2,6-di-t-butyl-4-methylpyridine) was filtered off. The ethereal filtrate was then evaporated and the residue was chromatographed on silica gel eluted with hexane/ethyl acetate=4/1 to provide the title compound (541 mg): LC-MS m/e 424(M+1), ¹H NMR (DMSO-d₆) δ 1.23 (d, 3H), 3.21 (t, 2H), 4.29 (q, 2H), 4.60 (t, 2H), 6.87 (d, 1H), 7.74 (dd, 1H), 7.84 (d, 1H).

### 4-(2-Chloro-phenylamino)-2-(2,3-dihydro-benzofuran-5-yl)-thiazole-5-carboxylic acid ethyl ester (Ida)

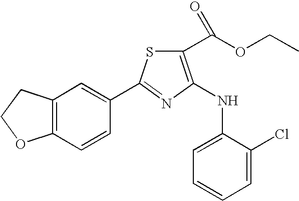

A mixture of 2-(2,3-dihydrobenzofuran-5-yl)-(4-trifluoromethanesulfonyloxy)thiazole-5-carboxylic acid ethyl ester (150 mg, 0.35 mmol), (R)-(+)-2,2′-bis(diphenylphosphino)-1,1′-binaphtyl (R—BINAP, 22 mg), Pd(OAc) 2 (8 mg), 2-chloroaniline (90 mg, 0.70 mmol), and CsCO₃ (185 mg, 0.52 mmol) in toluene (1.5 mL) was heated at 70 degree for 2 h under an argon atmosphere. The reaction mixture was directly purified by silica-gel column chromatography using AcOEt/hexane=1/5 to give the title compound (88 mg): LC-MS m/e 401 (M+1), ¹H NMR (CDCl₃) δ 1.40 (t, 3H), 3.30 (t, 2H), 5.39 (q, 2H), 4.68 (t, 2H), 6.85 (d, 1H), 6.94 (t, 1H), 7.32 (t, 1H), 7.40 (d, 1H), 7.82 (d, 1H), 7.87 (d, 1H), 8.63 (d, 1H), 9.73 (sbr, 1H).

### Example 9

### 4-(2-Chlorophenylamino)-2-(2,3-dihydrobenzofuran-5-yl)thiazole-5-carboxylic acid (Iea)

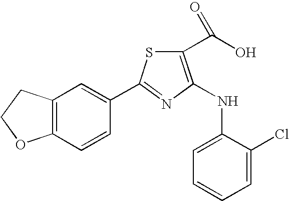

A mixture of 4-(2-chloro-phenylamino)-2-(2,3-dihydro-benzofuran-5-yl)-thiazole-5-carboxylic acid ethyl ester (80 mg), 1N-NaOH (1 mL), THF (1 mL) and EtOH (1 mL) was heated at 70 degree overnight. The mixture was neutralized with 1N—HCl to provide a precipitate. Filtration gave a yellowish solid (43 mg): LC-MS m/e 373 (M+1), ¹H NMR (DMSO-d₆) δ 3.29 (t, 2H), 4.64 (t, 2H), 6.92 (d, 1H), 6.97 (t, 1H), 7.39 (t, 1H), 7.48 (d, 1H), 7.83 (d, 1H), 7.91 (s, 1H), 8.59 (d, 1H), 10.20 (sbr, 1H).

### Example 10

### 4-(2-Chlorophenylamino)-2-(2,3-dihydrobenzofuran-5-yl) thiazole-5-carboxylic acid amide (Ieb)

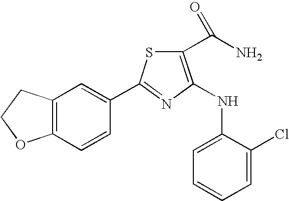

4-(2-Chloro-phenylamino)-2-(2,3-dihydrobenzofuran-5-yl)-thiazole-5-carboxylic acid (20 mg) and HBTU (27 mg) in DMF (1 mL) was added aq.NH₃ (3 drops) and Et₃N (3 drops). The mixture was stirred for 2 h at room temperature. The mixture was washed with H2O and the products were extracted with AcOEt. After evaporation, the residue was purified by silica-gel chromatography using AcOEt only to AcOEt/MeOH=100/2 as an eluant to give the title compound (16 mg): LC-MS m/e 372 (M+1), ¹H NMR (DMSO-d₆) δ 3.30 (t, 2H), 4.65 (t, 2H), 6.95 (d, 1H), 6.96 (t, 1H), 7.38 (t, 1H), 7.48 (d, 1H), 7.55 (sbr, 2H), 7.78 (d, 1H), 7.88 (s, 1H), 8.57 (d, 1H), 10.78 (s, 1H).

Compounds shown below were prepared in accordance with Example 8-10

### Example 11

### 4-(2-Chloro-5-fluorophenylamino)-2-(2,3-dihydrobenzofuran-5-yl) thiazole-5-carboxylic acid amide (Iec)

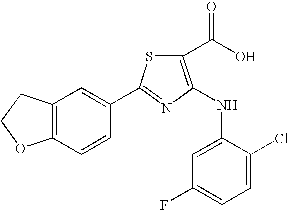

¹H NMR (DMSO-d₆) δ 3.29 (t, 2H), 4.64 (t, 2H), 6.84 (dt, 1H), 6.94 (d, 1H), 7.54 (dd, 1H), 7.81 (d, 1H), 7.88 (s, 1H), 8.45 (dd, 1H), 10.23 (sbr, 1H).

### Example 12

### 4-(2-Chlorophenylamino)-2-(4-methoxyphenyl) thiazole-5-carboxylic acid (Ied)

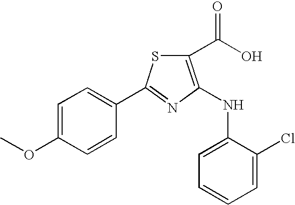

LC-MS m/e 361 (M+1), ¹H NMR (DMSO-d₆) δ 3.84 (s, 3H), 6.86 (t, 1H), 7.08 (d, 2H), 7.32 (t, 1H), 7.41 (d, 1H), 7.93 (d, 2H), 8.59 (d, 1H), 10.87 (sbr, 1H).

### Example 13

### 4-(5-Acetylamino-2-chlorophenylamino)-2-(4-methoxyphenyl) thiazole-5-carboxylic acid (Iee)

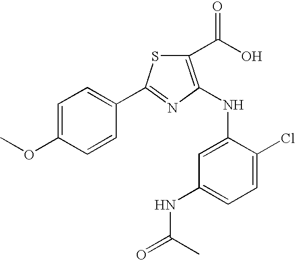

LC-MS m/e 418 (M+1), ¹H NMR (DMSO-d₆) δ 2.11 (s, 3H), 3.87 (s, 3H), 7.08 (d, 3H), 7.40 (d, 1H), 8.23 (d, 2H), 9.25 (a, 1H), 9.87 (brs, 1H), 10.11 (brs, 1H).

### Example 14

### 4-(5-Carbamoyl-2-chlorophenylamino)-2-(4-methoxyphenyl)thiazole-5-carboxylic acid (Ief)

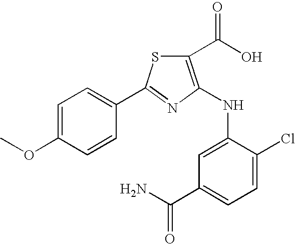

LC-MS m/e 404 (M+1), ¹H NMR (DMSO-d₆) δ 3.87 (s, 3H), 7.11 (d, 2H), 7.42 (s, 1H), 7.50 (dd, 1H), 7.60 (d, 1H), 8.05 (d, 2H), 8.06 (m, 1H), 9.20 (d, 1H), 9.85 (brs, 1H), 13.38 (brs, 1H).

### Example 15

### 4-(2-Chloro-5-sulfophenylamino)-2-(4-methoxyphenyl) thiazole-5-carboxylic acid (leg)

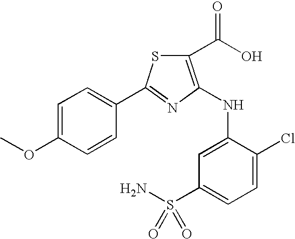

LC-MS m/e 441 (M+1), ¹H NMR (DMSO-d₆) δ 3.87 (s, 3H), 7.10 (d, 2H), 7.21 (dd, 1H), 7.45 (d, 1H), 8.06 (d, 2H), 9.08 (d, 1H), 9.83 (brs, 1H), 13.32 (brs, 1H).

### Example 16

### 4-(5-Amino-2-chlorophenylamino)-2-(4-methoxyphenyl) thiazole-5-carboxylic acid (Ieh)

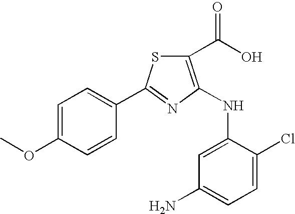

LC-MS m/e 376 (M+1), ¹H NMR (DMSO-d₆) δ 3.86 (s, 3H), 6.22 (d, 1H), 7.08 (d, 1H), 7.09 (d, 2H), 7.89 (d, 1H), 8.10 (d, 2H), 9.71 (brs, 1H).

### Example 17

### 4-(2-Chloro-4-methanesulfonylphenylamino)-2-(4-methoxyphenyl) thiazole-5-carboxylic acid (Iei)

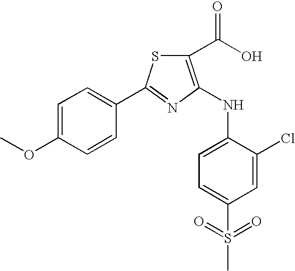

LC-MS m/e 439 (M+1), ¹H NMR (DMSO-d₆) δ 3.25 (s, 3H), 3.87 (s, 3H), 7.11 (d, 2H), 7.95 (dd, 1H), 8.04 (d, 1H), 8.08 (d, 2H), 8.90 (d, 1H), 10.25 (brs, 1H).

### Example 18

### 4-(4-Carboxy-2-chlorophenylamino)-2-(4-methoxyphenyl) thiazole-5-carboxylic acid (Iej)

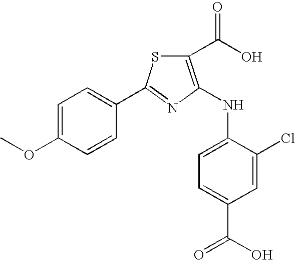

LC-MS m/e 405 (M+1), ¹H NMR (DMSO-d₆) δ 3.87 (9, 3H), 7.11 (d, 2H), 7.97 (m, 2H), 8.06 (d, 2H), 8.76 (d, 1H), 10.17 (sbr, 1H).

### Example 19

### 4-(2-Chlorophenylamino)-2-(pyridin-3-yl)thiazole-5-carboxylic acid (Iek)

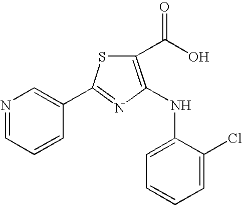

LC-MS m/e 332 (M+1), ¹H NMR (DMSO-d₆) δ 7.05 (t, 1H), 7.43 (t, 1H), 7.53 (d, 1H), 7.62 (dd, 1H), 8.44 (d, 1H), 8.60 (d, 1H), 8.77 (d, 1H), 9.27 (s, 1H), 9.83 (s, 1H).

### Example 20

### 4-(3,5-Dichloropyridin-4-ylamino)-2-(pyridin-3-yl) thiazole-5-carboxylic acid (Iel)

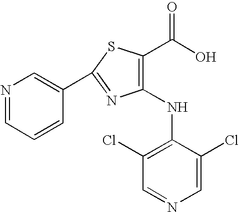

LC-MS m/e 367 (M+1), LC-MS m/e 367 (M+1), ¹H NMR (DMSO-d₆) δ 7.64 (dd, 1H), 8.18 (d, 1H), 8.69 (s, 1H), 8.70 (d, 2H), 9.02 (m, 2H), 13.4 (brs, 1H)

### Example 21

### 2-Pyridin-3-yl-4-(pyridin-3-ylamino)-thiazole-5-carboxylic acid (Iem)

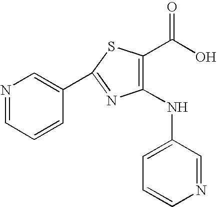

LC-MS m/e 299 (M+1), ¹H NMR (DMSO-d₆) δ 7.33 (dd, 1H), 7.56 (dd, 1H), 8.09 (d, 1H), 8.17 (d, 1H), 8.33 (d, 1H), 8.67 (d, 1H), 8.70 (d, 1H), 9.17 (d, 1H), 10.5 (sbr, 1H).

### Example 22

### 4-(2-Chlorophenylamino)-2-(pyridin-4-yl)-thiazole-5-carboxylic acid (Ien)

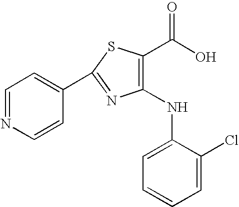

LC-MS m/e 332 (M+1), ¹H NMR (DMSO-d₆) δ 6.89 (t, 1H), 7.34 (t, 1H), 7.43 (dd, 1H), 7.91 (d, 1H), 8.58 (d, 1H), 8.72 (d, 2H), 10.95 (sbr, 1H).

### Example 23

### 4-[2-(3-Chlorophenyl)ethylamino]-2-(pyridin-4-yl) thiazole-5-carboxylic acid (Ieo)

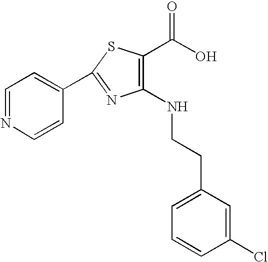

LC-MS m/e 360 (M+1), ¹H NMR (DMSO-d₆) δ 2.93 (t, 2H), 3.79 (q, 2H), 7.10 (tbr, 1H), 7.23 (m, 2H), 7.30 (m, 1H), 7.36 (s, 1H), 7.87 (d, 2H), 8.73 (d, 2M).

### Example 24

### 4-[2-(3-Chlorophenyl)ethylamino]-2-(pyridin-4-yl)-thiazole-5-carboxylic acid amide (Iep)

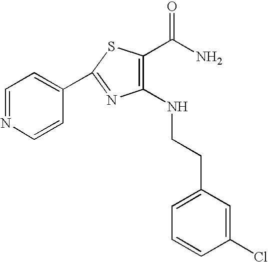

LC-MS m/e 359 (M+1), ¹H NMR (DMSO-d₆) δ 2.91 (t, 2H), 3.75 (q, 2H), 7.22-7.35 (m, 6H), 7.59 (t, 1H), 7.81 (d, 2H), 8.73 (d, 2H).

### Example 25

### 4-(2-Chloro-5-fluorophenylamino)-2-(pyridin-3-yl)thiazole-5-carboxylic acid (Ieq)

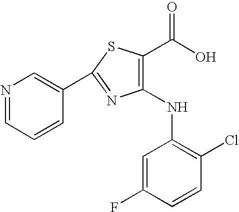

LC-MS m/e 350 (M+1), ¹H NMR (DMSO-d₆) δ 6.80 (dt, 1H), 7.53 (dd, 1H), 7.61 (dd, 1H), 8.37 (d, 1H), 8.45 (dd, 1H), 8.73 (dd, 1H), 9.20 (d, 1H).

### Example 26

### 4-(2-Chloro-5-fluoro-phenylamino)-2-(pyridin-3-yl)thiazole-5-carboxylic acid amide (Ier)

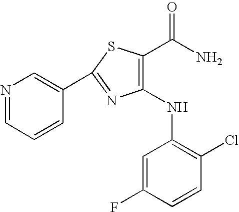

LC-MS m/e 349(M+1), ¹H NMR (DMSO-d₆) δ 6.48 (t, 1H), 7.53 (t, 1H), 7.65 (dd; 1), 7.6-8.0 (sbr, 2H), 8.34 (d, 1H), 8.43 (d, 1H), 8.77 (d, 1H), 9.17 (s, 1H), 10.97 (sbr, 1H).

### Example 27

### 2-(Pyridin-3-yl)-4-(2-trifluoromethyl-phenylamino)thiazole-5-carboxylic acid amide (Ies)

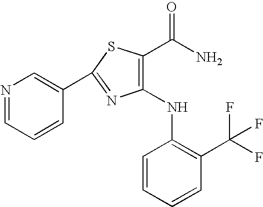

LC-MS m/e 365 (M+1), ¹H NMR (DMSO-d₆) δ 7.17 (t, 1H), 7.61 (dd, 1H), 7.6-8.0 (sbr, 2H), 7.69 (m, 2H), 8.32 (dt, 1H), 8.43 (d, 1H), 8.74 (dd, 1H), 9.16 (d, 1H), 10.74 (s, 1H).

### Example 28

### 4-(4-Chlorobenzylamino)-2-(pyridin-3-yl)thiazole-5-carboxylic acid (Iet)

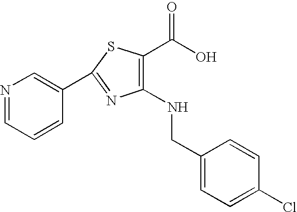

LC-MS m/e 346 (M+1), ¹H NMR (DMSO-d₆) δ 4.72 (d, 2H), 7.37 (d, 2H), 7.42 (d, 2H), 7.54 (dd, 1H), 7.60 (tbr, 1H), 8.27 (dt, 1H), 8.70 (dd, 1H), 9.10 (d, 1H).

### Example 29

### 4-(4-Chlorobenzylamino)-2-(pyridin-3-yl) thiazole-5-carboxylic acid (Ieu)

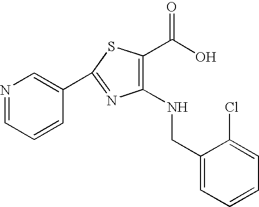

LC-MS m/e 346 (M+1), ¹H NMR (DMSO-d₆) δ 7.42 (m, 2H), 7.57 (m, 2H), 7.65 (dd, 1H), 7.69 (t, 1H), 8.38 (dt, 1H), 8.82 (dd, 1H), 9.22 (d, 1H)

### Biological Methods and Data

As demonstrated by the representative compounds of the present invention in Table 1, the compounds of the present invention have valuable pharmacological properties due to their potent ability to inhibit the hYAK3 and CK2 kinase enzyme.

Substrate phosphorylation assays were carried out as follows:

### YAK3 Scintillation Proximity Assays Using Ser164 of Myelin Basic Protein as the Phosphoacceptor

The source of Ser164 substrate peptide The biotinylated Ser164, S164A peptide(Biotinyl—LGGRDSRAGS*PMARR—OH), sequence derived from the C-terminus of bovine myelin basic protein (MBP) with Ser162 substituted as Ala162, was purchased from California Peptide Research Inc. (Napa, Calif.), and its purity was determined by HPLC. Phosphorylation occurs at position 164 (marked S* above). The calculated molecular mass of the peptide was 2166 dalton. Solid sample was dissolved at 10 mM in DMSO, aliquoted, and stored at −20° C. until use.

The source of hYAK3 enzyme: Glutathione-S-Transferase (GST)-hYak3-His6 containing amino acid residues 124-526 of human YAK3 (aa 124-526 of SEQ ID NO 2. in U.S. Pat. No. 6,323,318) was purified from baculovirus expression system in Sf9 cells using Glutathione Sepharose 4B column chromatography followed by Ni-NTA-Agarose column chromatography. Purity greater than 65% typically was achieved. Samples, in 50 mM Tris, 150 mM NaCl, 10% glycerol, 0.1% Triton, 250 mM imidazole, 10 mM β-mercapto ethanol, pH 8.0. were stored at −80° C. until use.

Kinase assay of purified hYAK3: Assays were performed in 96 well (Costar, Catalog No. 3789) or 384 well plates (Costar, Catalog No. 3705). Reaction (in 20, 25, or 40 μl volume) mix contained in final concentrations 25 mM Hepes buffer, pH 7.4; 10 mM MgCl₂; 10 mM β-mercapto ethanol; 0.0025% Tween-20; 0.001 mM ATP, 0.1 □Ci of [□-33P]ATP; purified hYAK3 (7-14 ng/assay; 4 nM final); and 4 μM Ser164 peptide. Compounds, titrated in DMSO, were evaluated at concentrations ranging from 50 μM to 0.5 nM. Final assay concentrations of DMSO did not exceed 5%, resulting in less than 15% loss of YAK3 activity relative to controls without DMSO. Reactions were incubated for 2 hours at room temperature and were stopped by a 75 ul addition of 0.19 μg Streptavidin Scintillation Proximity beads (Amersham Pharmacia Biotech, Catalog No. RPNQ 0007) in PBS, pH 7.4, 10 mM EDTA, 0.1% Triton X-100, 1 mM ATP. Under the assay conditions defined above, the Km(apparent) for ATP was determined to be 7.2+/−2.4 μM.

TABLE 1

| Compound No. | pIC₅₀ values |
|---|---|
| Ibd | ++ |
| Ibf | + |

Legend

| | |
|---|---|
| pIC₅₀ values | Symbol |
| 6.99-6.00 | ++ |
| 5.99-5.00 | + |

pIC₅₀ = −log₁₀(IC₅₀)

Compounds of the present invention were also tested for casein kinase 2 alpha (CK2α) inhibitory activity in substrate phosphorylation assays. This assay examines the ability of small molecule organic compounds to inhibit the serine phosphorylation of a peptide substrate.

### Kinase Assays Using Peptide Phosphoacceptor

The source of Peptide

The biotinylated peptide (Biotin—GGRRRDDDS*DDD-OH), was purchased from Biosource International, Inc. (Camarillo, Calif.), and its purity was determined by HPLC. Phosphorylation of serine (S*) occurs under assay conditions described. The calculated molecular mass of the peptide was 1713 dalton. Solid sample was dissolved at 10 mM in DMSO, aliquoted, and stored at −20° C. until use.

The Source of Enzyme:

Recombinant expression of casein kinase 2 alpha (CK2α), using baculovirus-based vectors in Sf21 insect cells and purified by P11 phosphocellulose (Whatman) chromatography of the clarified cell lysate, produced constitutively active enzyme. CK2α was co-expressed with CK2β. Samples were stored at −20° C. in the presence of 50% glycerol until use.

Kinase Assay of Purified CK2α:

Assays were performed in 384 well plates (Costar, Catalog No. 3705). Compounds first were delivered to the plate in one microliter DMSO, followed by reaction mix (in 40 ul volume), which contained in final concentrations 25 mM Hepes buffer, pH 7.4; mM MgCl₂; 150 mM NaCl; 1 mM β-mercapto ethanol; 0.0025% Tween-20; 0.001 mM ATP, 0.1 μCi of [γ-33P]ATP; purified CK2α (6 ng/assay), and 0.001 mM peptide. Reactions were incubated for 3 hours at room temperature and were stopped by a 0.040 ml addition of 0.16 mg Streptavidin Scintillation Proximity beads (in PBS, pH 7.4, 10 mM EDTA, 0.1% Triton X 100, 1 mM ATP). Quenched reactions remained overnight before 1 minute centrifugation (at 500×g), followed by counting in a Packard TopCount.

The data for dose responses were plotted as % Inhibition, calculated with the data reduction formula 100*(1−[(U1−C2)/(C1−C2)]), versus concentration of compound, where U is the unknown value, C1 is the average control value obtained for DMSO, and C2 is the average control value obtained for 0.05M EDTA Data were fitted to the curve described by: y=((Vmax*x)/(K+x)) where Vmax is the upper asymptote and K is the IC50. The results for each compound were recorded as pIC50 calculated as follows: pIC50=−Log 10(K) in Table 2.

TABLE 2

| Compound No. | pIC₅₀ values |
|---|---|
| Ief | +++ |
| Ieg | ++ |
| Ieb | + |

Legend

| | |
|---|---|
| pIC₅₀ values | Symbol |
| 7.00-7.99 | +++ |
| 6.99-6.00 | ++ |
| 5.99-5.00 | + |

pIC₅₀ = −log₁₀(IC₅₀)

### Utility of the Present Invention

The above biological data clearly shows that the present compounds are useful for treating or preventing disease states in which hYAK3 proteins are implicated, especially diseases of the erythroid and hematopoietic systems, including anemias due to renal insufficiency or to chronic disease, such as autoimmunity, HIV or cancer and drug-induced anemias, myelodysplastic syndrome, aplastic anemia and myelosuppression; cytopenia.

The present method is especially useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

The present invention provides a method of enhancement of normal red blood cell numbers. Such enhancement is desirable for a variety of purposes, especially medical purposes such as preparation of a patient for transfusion and preparation of a patient for surgery.

The above biological data also clearly shows that the present compounds are useful for treating or preventing disease states in which CK2 proteins are implicated, especially cancers and viral infections.

1. A compound of the formula I, or a salt, solvate, or a physiologically functional derivative thereof 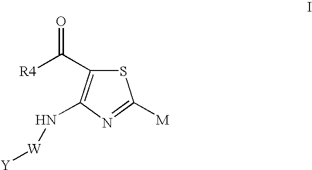
in which
M is a radical of the formula 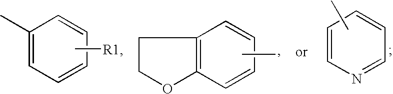
Y is a radical of the formula 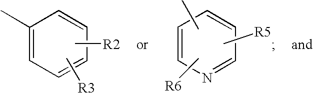
R1, R2 and R3 are independently hydrogen, —NH₂, halogen, —OC₁₋₆ alkyl, —CF₃, —N(C═O)CH₃, —(C═O)OH, —CF₃, —(C═O)NH₂, —SO₂CH₃, —SO₂OH, or —C₁₋₆alkyl;
W is —(CH₂)ₙ—, in which n is 0 to 2; and
R4 is —NH₂, or —OH; and
R5 and R6 are independently hydrogen or halogen.

2. A compound of Formula I of claim 1 in which M is a radical of the formula 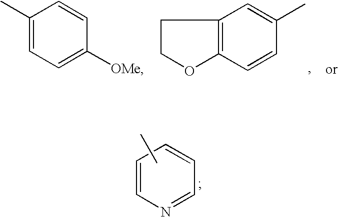
Y is a radical of the formula 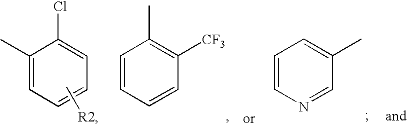
in which R2 is hydrogen, —NH₂, halogen, —OC₁₋₆alkyl, —CF₃, —N(C═O)CH₃, —(C═O)OH, —CF₃, —(C═O)NH₂, —SO₂CH₃, —SO₂OH, or —C₁₋₆alkyl.

3. A method of inhibiting hYAK3 and/or CK2 in a mammal; comprising, administering to the mammal a therapeutically effective amount of a compound of formula I of claim 1, or a salt, solvate, or a physiologically functional derivative thereof.

4. A pharmaceutical composition including a therapeutically effective amount of a compound of formula I of claim 1, or a salt, solvate, or a physiologically functional derivative thereof and one or more of pharmaceutically acceptable carriers, diluents and excipients.

5. A method of treating or preventing diseases of the erythroid and hematopoietic systems selected from the group consisting of: neutropenia; cytopenia; anemias, including anemias due to renal insufficiency or to a chronic disease, such as autoimmunity, HIV or cancer, and drug-induced anemias; and myelosuppression; comprising administering to a mammal a therapeutically effective amount of a compound of formula I of claim 1, or a salt, solvate, or a physiologically functional derivative thereof and one or more of pharmaceutically acceptable carriers, diluents and excipients.

6. A method of treating or preventing cancer or viral infections; comprising administering to a mammal a therapeutically effective amount of a compound of formula I of claim 1, or a salt, solvate, or a physiologically functional derivative thereof and one or more of pharmaceutically acceptable carriers, diluents and excipients.

7. A compound of formula I of claim 1 selected from the group consisting of
4-anilino-5-carboxyl-2-(4-methoxyphenyl)thiazole;
4-anilino-5-methoxycarbonyl-2-(4-methoxyphenyl) thiazole;
5-aminocarbonyl-2-(3-methoxyphenyl)-4-(2-trifluoromethylanilino)thiazole;
5-methoxycarbonyl-2-(3-methoxyphenyl)-4-(2-trifluoromethylanilino) thiazole;
5-carboxyl-2-(4-methoxyphenyl)-4-(2-trifluoromethyl)anilinothiazole;
5-aminocarbonyl-2-(3-methoxyphenyl)-4-(2-trifluoromethylanilino)thiazole;
5-carboxyl-4-(3-fluoroanilino)-2-(4-methoxyphenyl)thiazole;
5-carboxyl-2-(4-methoxyphenyl)-4-(2-trifluoromethylanilino)thiazole;
4-anilino-5-carboxyl-2-(3-methoxyphenyl)thiazole;
5-carboxyl-4-(2-fluoroanilino)-2-(3-methoxyphenyl)thiazole;
4-benzylamino-5-methoxycarboxyl 2-(4-methoxyphenyl)thiazole
4-(2-chloro-phenylamino)-2-(2,3-dihydro-benzofuran-5-yl)-thiazole-5-carboxylic acid ester;
4-(2-chlorophenylamino)-2-(2,3-dihydrobenzofuran-5-yl) thiazole-5-carboxylic acid;
4-(2-chlorophenylamino)-2-(2,3-dihydrobenzofuran-5-yl) thiazole-5-carboxylic acid amide;
4-(2-chloro-5-fluorophenylamino)-2-(2,3-dihydrobenzofuran-5-yl) thiazole-5-carboxylic acid amide;
4-(2-chlorophenylamino)-2-(4-methoxyphenyl) thiazole-5-carboxylic acid;
4-(5-acetylamino-2-chlorophenylamino)-2-(4-methoxyphenyl) thiazole-5-carboxylic acid;
4-(5-carbamoyl-2-chlorophenylamino)-2-(4-methoxyphenyl) thiazole-5-carboxylic acid;
4-(2-chloro-5-sulfophenylamino)-2-(4-methoxyphenyl) thiazole-5-carboxylic acid;
4-(5-amino-2-chlorophenylamino)-2-(4-methoxyphenyl) thiazole-5-carboxylic acid;
4-(2-chloro-4-methanesulfonylphenylamino)-2-(4-methoxyphenyl) thiazole-5-carboxylic acid;
4-(4-carboxy-2-chlorophenylamino)-2-(4-methoxyphenyl) thiazole-5-carboxylic acid;
4-(2-chlorophenylamino)-2-(pyridin-3-yl) thiazole-5-carboxylic acid;
4-(3,5-dichloropyridin-4-ylamino)-2-(pyridin-3-yl) thiazole-5-carboxylic acid;
2-pyridin-3-yl-4-(pyridin-3-ylamino)-thiazole-5-carboxylic acid;
4-(2-chlorophenylamino)-2-(pyridin-4-yl)-thiazole-5-carboxylic acid;
4-[2-(3-chlorophenyl)ethylamino]-2-(pyridin-4-yl) thiazole-5-carboxylic acid;
4-[2-(3-chlorophenyl)ethylamino]-2-(pyridin-4-yl)-thiazole-5-carboxylic acid amide;
4-(2-chloro-5-fluorophenylamino)-2-(pyridin-3-yl) thiazole-5-carboxylic acid;
4-(2-chloro-5-fluoro-phenylamino)-2-(pyridin-3-yl) thiazole-5-carboxylic acid amide;
2-(pyridin-3-yl)-4-(2-trifluoromethyl-phenylamino)thiazole-5-carboxylic acid amide;
4-(4-chlorobenzylamino)-2-(pyridin-3-yl)thiazole-5-carboxylic acid; and
4-(4-chlorobenzylamino)-2-(pyridin-3-yl)thiazole-5-carboxylic acid.
